(19) United States
(12) Patent Application Publication
(10) Pub. No.: US 2012/0107925 A1
(43) Pub. Date: May 3, 2012

(54) DEVICES FOR SEPARATING CELLS AND METHODS OF USING THEM

(76) Inventors: Mo Huang Li, Tucson, AZ (US); Jackie Y. Ying, Singapore (SG); Jiashen Wei, Singapore (SG); Chye Cheong Wai, Singapore (SG)

(21) Appl. No.: 13/257,972

(22) PCT Filed: Mar. 19, 2010
(86) PCT No.: PCT/SG2010/000104

§ 371 Date: Jan. 6, 2012

(30) Foreign Application Priority Data
Mar. 20, 2009 (SG) 2009019373

(51) Int. Cl. C12N 5/00; C12M 1/00

(52) U.S. Cl. 435/325; 435/283.1

(57) ABSTRACT

The present invention refers to a device for separating cells of a defined size from a sample and to a system comprising such devices and further components. Such devices can comprise different components, such as inlet module, intermediate module, outlet module and microsieve. The present invention also refers to methods of operating such devices and uses thereof.

### CROSS-REFERENCE TO RELATED APPLICATIONS

This application claims the benefit of priority of SG application No. 200901937-3, filed Mar. 20, 2009, the contents of it being hereby incorporated by reference in its entirety for all purposes.

### FIELD OF THE INVENTION

The present invention refers to the field of laboratory equipment used for isolating specific cells from samples of a patient.

### BACKGROUND OF THE INVENTION

The isolation of specific components from liquid samples has become increasingly important not only for research purposes but also for diagnostics in clinical laboratories. In particular for clinical applications systems are needed which can determine the presence or absence of certain components in samples obtained from a patient in a fast and reliable manner which allows a clinician to make a diagnosis and determine the further treatment of a patient. Such systems are also need in research laboratories.

For example, circulating tumor cells (CTCs) are very rare in peripheral blood of cancer patients, as few as one cell per 10⁹ haematologic cells in the blood of cancer patients with metastatic cancer. The number of these cells has been shown to correlate with the disease development, and represents a potential alternative to invasive biopsies for cancer metastasis analysis. Several technologies have been developed to isolate the CTCs from cancer patients' blood samples, such as density gradient centrifugation, immunomagnetic method (MACS), ferrofluid method (CellSearch™), and cell filtration by polymer membrane. However, these methods are either requiring a prolonged process time (>30 min) or with limited extraction efficiency. In addition, methods utilizing conjugated antibodies specific to epithelial antigens (EpCAM, cytokeratin) could give false positive results.

Therefore, there is a need to develop systems for isolating certain components from patients samples.

### SUMMARY OF THE INVENTION

In a first aspect the present invention refers to a device for separating cells of a defined size from a sample. The device comprises or consists of an inlet module having an inlet; an outlet module having an outlet; an intermediate module having a through-hole and being arranged between the inlet module and the outlet module; and a microsieve having micropores for retaining cells of a defined size. The components of the device can be designed so that the inlet module, the intermediate module and the outlet module are removably and fluidly connected to each other. Furthermore, the microsieve can be arranged between the intermediate module and the outlet module. The outlet module can be adapted to exert a negative pressure at the outlet thereof. A channel which is formed by each of the pores of the microsieve through the microsieve has a length of at least 50 μm or in other words the pore length is about at least 50 μm. In one embodiment the length is between about 150 μm to about 250 μm.

In one embodiment, the inlet module can comprise a protrusion which is arranged at the side of the inlet module opposite the inlet and wherein the protrusion is a continuation of the inlet and is adapted to contact the surface of the intermediate module around the through-hole of the intermediate module when assembled.

In one embodiment, the intermediate module can have the shape of a cone. The through-hole of the intermediate module can be arranged at the bottom of the cone. The cones tip with the through-hole can face the microsieve.

In a further embodiment, the intermediate layer may define a filtering area on the microsieve. The size of the filtering area is defined by the size of the through-hole of the intermediate layer.

In still another embodiment, the outlet module can comprise a top surface having a recess arranged within the top surface. The size of the recess may be adapted for holding the microsieve. Furthermore, the outlet of the outlet module can be arranged at the bottom of the recess, such as at the centre of the recess.

In another embodiment, the outlet in the recess of the outlet module may be positioned opposite the opening of the intermediate layer.

In another embodiment, the outlet module may comprise a side wall with an orifice. The orifice is positioned outside the recess and is connectable to a device exerting a negative pressure.

In another embodiment, the side wall of the outlet module may be adapted to be connectable to a container.

In another aspect, the present invention refers to a device for separating cells of a defined size from a sample. The device can comprise or consist of an inlet module having an inlet; an outlet module having an outlet; an intermediate module having a through-hole. The intermediate module may be arranged between the inlet module and the outlet module. The device can further comprise a microsieve having micropores for retaining cells of a defined size. The microsieve can be arranged between the inlet module and the intermediate module. Furthermore, the inlet module, the outlet module, and the intermediate module are removably and fluidly connected to each other. A channel which is formed by each of the pores of the microsieve through the microsieve has a length of at least 50 μm or in other words the pore length is about at least 50 μm. In one embodiment the length is between about 150 μm to about 250 μm.

In one embodiment, this device can comprise a spacer comprising a through-hole. The spacer can be arranged between the microsieve and the inlet module. The spacer may define a filtering area on the microsieve. The size of the filtering area can be defined by the size of the through-hole of the spacer.

The intermediate module can comprise a recess for holding the microsieve. Furthermore, the recess can be dimensioned to hold the microsieve and the spacer.

The filtering area in the devices described herein can have a maximal diameter of between about 0.5 to 20 mm or between about 2 to 5 mm.

The thickness of the microsieve used in any of the devices described herein can be between about 50 μm to about 1000 μm or about 150 μm.

The maximal horizontal extension of the microsieve can be between about 1 mm to about 3 cm or about 1.5 cm. Furthermore, the micropores can be spaced apart from each other in a uniform or non-uniform pattern.

In another embodiment, the diameter of each of the micropores is between about 2 μm to about 20 μm or about 10 μm. Depending on the cell to be separated from a liquid sample, the diameter of each of the micropores is adapted to the size of the cell to be separated.

In another embodiment, the maximal distance from the center of one micropore to the center of another micropore is between about 2 μm to about 100 μm or about 12 μm.

In still another embodiment, the devices referred to herein further comprise positioners arranged at the contact areas between the modules to fix the position of the modules relative to each other when assembling the modules.

In another aspect the present invention refers to a system. This system can comprise a cell separation device described herein, a container connected to the outlet module of the device for collecting waste and an apparatus for exerting a negative pressure. The device for exerting a negative pressure can be connected to an orifice of the device adapted to be connectable to the apparatus for exerting a negative pressure. The system can further comprise a liquid source for holding a liquid sample; wherein the liquid source is fluidly connected to the inlet of the device.

In a further aspect, the present invention refers to a method of separating cells of a defined size from a liquid sample. The method can comprise filtering a liquid sample suspected to comprise a cell to be separated through an inlet of one of the devices described herein. In a further embodiment, the method further comprises removing the separated cells from the microsieve.

In one embodiment, the liquid sample is a blood sample, such as a whole blood sample.

In another embodiment, the cells to be detected are circulating tumor cells (CTCs).

In another aspect, the present invention refers to the use of a device described herein for filtering blood, for example to separate circulating tumor cells (CTCs) from blood, such as a whole blood liquid sample.

### BRIEF DESCRIPTION OF THE DRAWINGS

The invention will be better understood with reference to the detailed description when considered in conjunction with the non-limiting examples and the accompanying drawings, in which:

FIG. 1(A) to (C) show the components of a cell separating device according to a first aspect of the invention.

FIG. 2 shows technical drawings and schematics of an inlet module 1 of a cell separating device described herein. The dimension in FIG. 2 is in millimeter (mm).

FIG. 3 shows technical drawings and schematics of an intermediate module 2 of a cell separating device described herein. The dimension in FIG. 3 is in millimeter (mm).

FIG. 4 shows technical drawings and schematics of an outlet module 3 of a cell separating device described herein. The dimension in FIG. 4 is in millimeter (mm).

FIG. 5(A) to (D) shows the components of a further cell separating device comprising an inlet module 4, a spacer 5, a microsieve 6, an intermediate module 7 and an outlet module 8.

FIG. 6(A) to (C) show SEM pictures. (A) Silicon microsieve filter with uniform pore structure. (B) Cross section of silicon microsieve with smooth vertical through holes. (C) Commercial glass capillary array filter with HepG2 (hepatocellular carcinoma) cell captured. The filter has pore size of 10 μm with 12 μm pitch.

FIG. 7(A) shows the experimental setup for flow rate measurement. (B) Flow rate versus the filtration time (in seconds (s)) with variant filtration area 61 diameter on microsieve 6 of 2 to 5 mm. The filtration area 61 is defined by the opening of the spacer 5. Measured flow rate with undiluted rabbit whole blood (5 ml) spiked with 10⁴ of HepG2 cells is plotted together for reference. The insert shows the flow rate profiles of initial phase (<15 s). The arrow indicates the point of whole blood contacting with microsieve membrane.

FIG. 8 shows a diagram illustrating the recovery rate of HepG2 (10, 50 and 100 cells) spiked in 1-ml rabbit whole blood sample. The average recovery rate is >90%.

FIG. 9 shows capture of spiked HepG2 tumor cells on glass capillary microsieve membrane. (a) Bright field image. (b) Fluorescence image of HepG2 cells (bright dots). (c) Fluorescence image with cell nucleus stained with DAPI (bright dots). (d) Merge of fluorescence images showing the boundary of HepG2 and pore array (the background honeycomb structure).

FIG. 10 shows another diagram illustrating the recovery rate of HepG2 (5-200 cells/ml) diluted in 1× phosphate buffered saline. The average recovery rate is >94%.

FIG. 11 shows another fluorescence image of a filtrated whole blood sample of rat with lung cancer. (A) Nucleus of CTCs stained with DAPI (visible bright spots in the image). (B) Merge of bright field and fluorescence images showing the boundary of CTCs (hexagons appearing bright) and pore array (the background honeycomb structure).

FIG. 12 illustrates the operation principle of isolating cells, such as circulating tumor cells (CTCs) enrichment using a microsieve comprised in a cell separation device described herein.

FIG. 13 shows different applications including the cell separation devices described herein.

FIG. 14 illustrates the fabrication process for a microsieve used in a cell separation device described herein.

### DETAILED DESCRIPTION OF THE PRESENT INVENTION

In a first aspect the present invention refers to a device for separating cells of a defined size from a sample. The device comprises or consists of an inlet module 1 having an inlet 14; an outlet module 3 having an outlet 35; an intermediate module 2 having a through-hole 21 and being arranged between the inlet module 1 and the outlet module 3; and a microsieve 6 having micropores for retaining cells of a defined size. The components of the device can be designed so that the inlet module 1, the intermediate module 2 and the outlet module 3 are removably and fluidly connected to each other. Furthermore, the microsieve 6 can be arranged between the intermediate module 2 and the outlet module 3. The outlet module 3 can be adapted to exert a negative pressure at the outlet thereof. A channel which is formed by each of the pores of the microsieve through the microsieve has a length 604 of at least 50 μm or in other words the pore length is about at least 50 μm. The specific length of these channels provides a fluidic flow condition that is similar to the conditions of in vivo blood flow in arterioles.

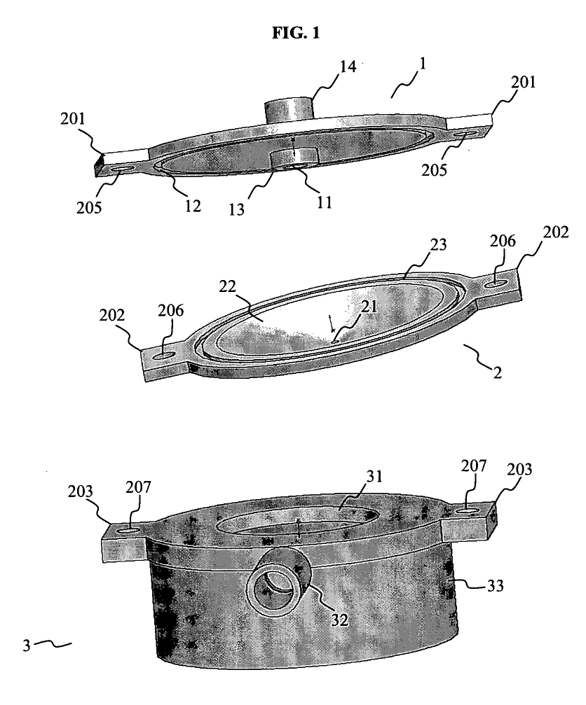

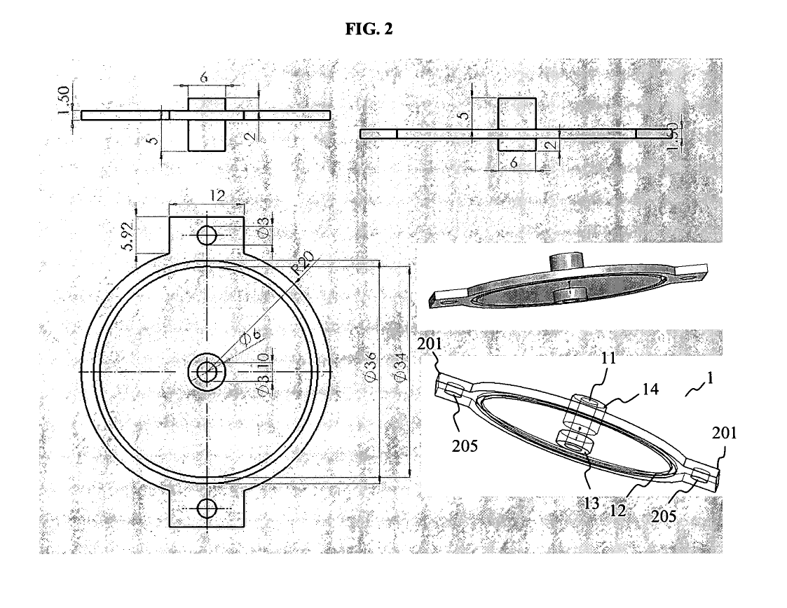

An exemplary embodiment of such a device is illustrated for example in FIGS. 1 to 4. The cell separation device shown in detail FIGS. 1 to 4 comprises an inlet module 1, and intermediate module 2 and an outlet module 3. The three different modules can be connected to each other so that the fluid flows in a vertical straight line starting from the inlet module 1 down to the outlet module 3 through the cell separation device. Avoiding turns and horizontal flow decreases the flow resistance. Thus, the openings 11, 21, 35 of the different modules 1, 2 and 3 are aligned along a vertical axis through the cell separation device when assembled.

Thus, a liquid sample flows through the opening 11 of the inlet 14 into through the inlet module 1 to the intermediate module 2. From the intermediate module 2 the liquid flows through the opening 21 of the intermediate module 2 through a microsieve 6 (not shown in FIGS. 1 to 4). After the liquid is filtered through the microsieve the filtered liquid exits the cell separation device through the outlet 35 of the outlet module 3.

To further increase the flow rate through the cell separation device shown in FIGS. 1 to 4, the inlet module 1 can comprise a protrusion 13 which is a continuation of the inlet 14 and which abuts at the surface of the intermediate module 2 around the through-hole 21 of the intermediate module 2. The protrusion either abuts the surface around the through-hole 21 or its stops short before it, i.e. about 0.5 to 1.5 mm above the surface of the intermediate module around the opening 21. The protrusion avoids that the liquid sample flowing from the inlet module 1 to the intermediate module 2 spreads out. The inner diameter of the opening 11 of the protrusion 13 can be between about 1 to 10 mm or between about 2 to 6 mm.

To further avoid that the liquid sample spreads out, the intermediate module 2 of the cell separation device can also comprise a cone shape or funnel shape 22 with the through-hole 21 forming the central point of the cone or funnel. A cone like shape means that the surface of the intermediate module forms a slope starting from a higher level at the outer boarder of the cone of the intermediate module 2 towards the lower leveled central point. The surface of the intermediate module 2 slopes towards the central tip of the cone comprising the through-hole 21 at an angle of at least 5° or between about 5° to 20°. This means for the dimensions of the protrusion 13 that the height of the protrusion 13 is adapted to a sufficient length so that the end of the protrusion abuts or almost abuts the surface of the cone around the through-hole 21.

Using an intermediate module 2 with a cone also avoids that the liquid flowing through the device spreads out. Even if it spreads out it will be drawn towards the central point of the intermediate module 2 via gravitation and/or the suction force of a vacuum applied to the device. The cone shape of the intermediate module further enables the practitioner to place the device under a light microscope after removing the inlet module. The flat cone can be adapted to fit the shape of an objective of a microscope to get closer to the microsieve carrying the separated cells after filtering for imaging of those cells. The microsieve is arranged directly below the through-hole 21 of the intermediate module 2.

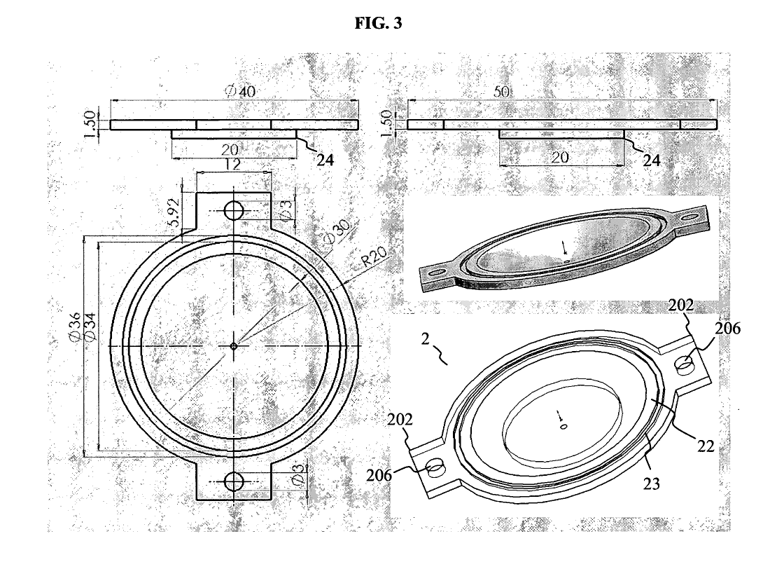

The microsieve 6 can be arranged between intermediate module 2 and outlet module 3. The microsieve 6 can be arranged directly at the surface of the outlet module 3 or within a recess 31 which is adapted to house the microsieve. Thus, the depth and the maximal depth of the recess 31 are adapted to fit the microsieve. Furthermore, the recess 31 can be even deeper, i.e. deeper than the maximal height of the microsieve, to allow the intermediate module 2 with the cone 22 to fit tightly onto the outlet module 3. As illustrated in FIG. 3, due to the cone 22, the intermediate module 2 can comprise an extension 24 which protrudes towards the outlet module 3. The extension or protrusion 24 can also be housed in the recess 31 of the outlet module 3. Thus, the recess 31 of the outlet module 3 can be adapted to house the microsieve 6 as well as the extension 24 of the intermediate module.

The microsieve can comprise an array of well ordered micropores as will be described in more detail below. The micropores can cover the entire microsieve or only a part thereof. The effective filtering area 61 is defined by the size of the through-hole 21 of the intermediate module. Thus, the intermediate module 2 with the cone shape and the central opening at the bottom of the cone also serves the purpose to define the filtering area 61 at the surface of the microsieve. To avoid that the liquid spreads over the entire microsieve after passing through the through-hole 21 of the intermediate module 2, the tip of the cone 22 with the through-hole 21 can abut or almost abut the surface of the microsieve to define the filtration area 61. Thus, irrespective of the size of the microsieve, the filtration area is self-defined by the size of the through-hole 21. In one embodiment, the maximal dimension of the through-hole is between about 0.5 to 10 mm. In another embodiment, the maximal dimension of the through-hole is between about 1 to 5 mm. The diameter of the cone 22 of the intermediate module 2 can be between about 10 to 40 mm but can also exceed this range depending on the overall size of the intermediate module. The through-hole 21 of the microsieve 6 can have any shape. In one example the shape is the shape of a square or a rectangle. In another embodiment, it has the shape of an oval or a circle. Irrespective of the size, the size of the through-hole 21 can cover an area of between about 1 to 100 mm², or between about 10 to 50 mm², or between about 7 to 28 mm². In one embodiment the size of the through-hole 21 is about 20 mm². The size of the filtration area 61 can also be adapted depending on the volume of the liquid sample used.

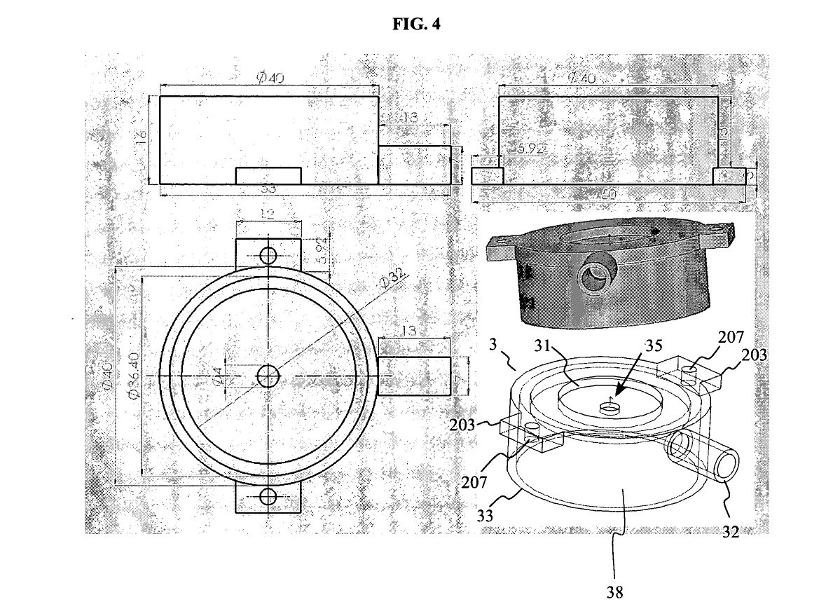

The outlet of the outlet module can be arranged within the center of the recess or off the center of the recess. As illustrated in FIG. 4, the outlet 35 is arranged at the center of the recess directly below the filtration area 61 defined by the through-hole 21 of the intermediate module.

The outlet module can further comprise an orifice 32 which is connectable to a device which exerts a negative pressure, i.e. a vacuum at the outlet 35 of the outlet module. Applying a vacuum to the outlet module 3 provides the driving force to filter the liquid sample faster through the cell separation device. The orifice or connector 32 can be arranged at the side wall of the outlet module.

The shape of the modules 1, 2, 3 can be individually adapted. They can all have the same form or different forms as long as they can be connected to each other to allow a flow of the liquid sample through the cell separation device. Thus, the modules 1, 2, 3 are fluidly connected to each other. As illustrated in FIGS. 1 to 4 the outer shape of the modules 1, 2, 3 is round. The shape can also be square like or rectangular. The shape of every module 1, 2, 3 of the cell separation device or at least the outlet module 3 can be adapted to be connectable to a container in which the liquid, which passed through the device, is collected.

Therefore, the outlet module can comprise an extended side wall 33 which is adapted to be connectable to a container. For example, the side wall can comprise threads on the inside which allows it to screw the cell separation device to a container, such as a flask or centrifuge tube (see e.g. FIG. 13). It can also comprise a snap-fit connection instead of a thread which would allow fitting the cell separation device on containers with a slightly varying opening size.

The modules 1, 2, 3 of the cell separation device can be connected to each other via connectors 201, 202, 203 which are protruding from the side wall of the modules 1, 2, 3 of the cell separation device. The connectors 201, 202, 203 can be arranged at opposite sides of the respective module 1, 2, 3 and provide openings 205, 206, 207 for inserting fixation means known in the art, such as screws. Instead of using openings for fixation means the modules 1, 2, 3 can also be connected to each other using snap-fit connectors catch the respective underlying module.

To ensure a water-tight sealing, the modules 1, 2, 3 can further provide trenches (e.g. 12, 23) running around the outer perimeter of the modules 1, 2, 3. Water repellent isolation materials can be fit into those trenches to provide a water tight closure when the modules 1, 2, 3 are assembled. Such an isolation material can be any available material, such as a polymer o-ring or gasket.

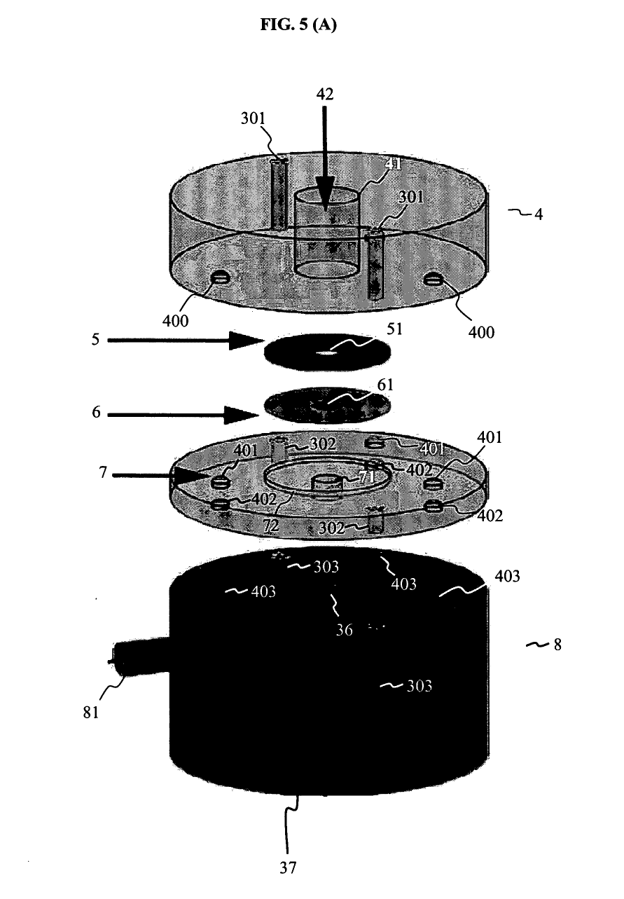

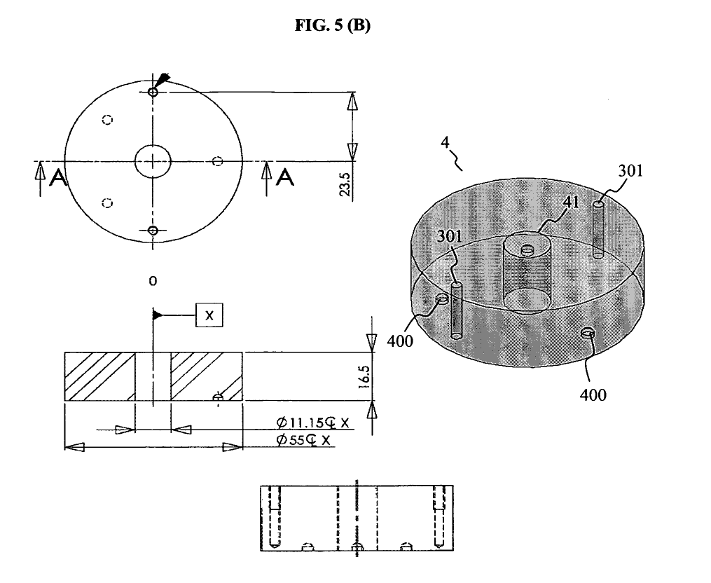

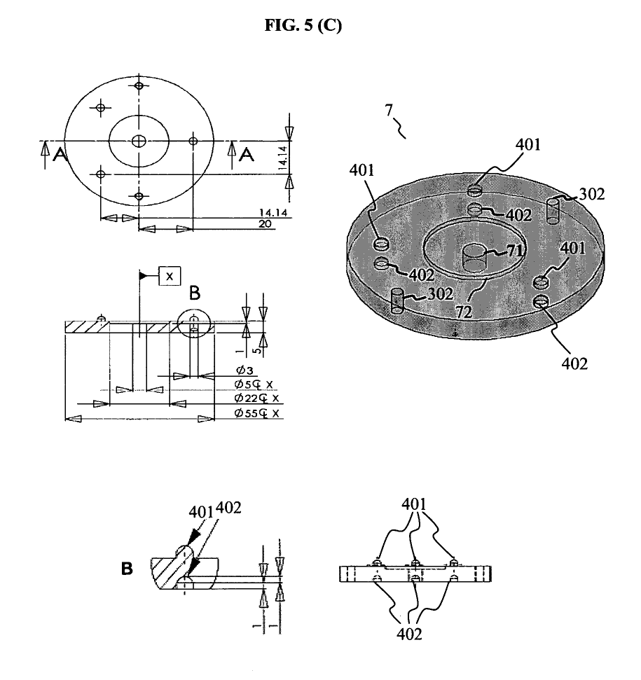

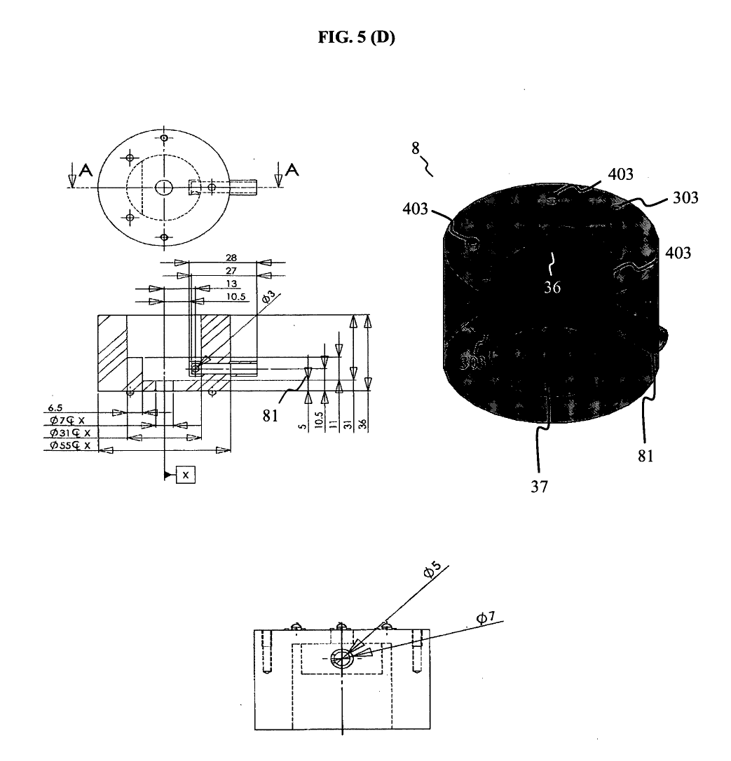

A further embodiment of a cell separation device is illustrated in FIG. 5. The cell separation device illustrated in FIG. 5 comprises an inlet module 4 having an inlet 41, an outlet module 8 having an outlet 36 and an intermediate module 7 having a through-hole 71. The intermediate module 7 may be arranged between the inlet module 4 and the outlet module 8. The device can further comprise a microsieve 6 having micropores for retaining cells of a defined size. The microsieve 6 can be arranged between the inlet module 4 and the intermediate module 7. Furthermore, the inlet module 4, the outlet module 8, and the intermediate module 7 are removably and fluidly connected to each other.

Other than in the embodiment illustrated in FIGS. 1 to 4, the microsieve 6 is arranged between the inlet module 4 and the intermediate module 7. A filtration area 61 can be defined by a spacer that can be arranged between inlet module 4 and microsieve 6. In case no spacer is comprised in the cell separation device shown in FIG. 5, the filtration area 61 is defined by the opening 42 of the inlet 41 which contacts the microsieve 61. The inlet 41 can have a conical shape which allows varying the size of the opening of the inlet facing the microsieve. In case of a round opening this would mean that the first opening of the inlet 41 connectable to the liquid source is larger than the diameter of the second opening of the inlet 41 facing the microsieve or vice versa. The diameter of a round second opening would then define the size of the filtration area 61 at the surface of the microsieve.

The inlet module 4 can also comprise a protrusion 13 like the inlet module 1 of the device illustrated in FIGS. 1 to 4. In this case the opening of the protrusion facing the microsieve would define the filtration area on the microsieve 6. As mentioned above, the micropores comprised in the microsieve can exceed the filtration area and in one embodiment extend about the entire size of the microsieve 6.

The spacer 5 can have a size and shape which equals the size and the shape of the inlet module and/or the intermediate module. In another embodiment, the spacer 5 can have a size and shape which equals the size and the shape of the microsieve 61. The size of the spacer 5 can also be smaller than the size of the microsieve as long as it provides an opening 51 which defines a filtration area 61 at the surface of the microsieve 6. The spacer can be a gasket and can be made of a flexible polymeric material known in the art. Thus, the spacer would not only define the filtration are but also provide a liquid tight sealing.

With reference to FIG. 5, the cell separation device comprises an inlet module 4 which can be of any shape and form as already described for the inlet module 1 of the embodiment shown in FIGS. 1 to 4. The inlet 41 can extend into a connector extending towards the side of the inlet module which is connectable to a liquid source. The cell separation device shown in FIG. 5 can further comprise a spacer. 5 and a microsieve 6 as just described.

Further comprised can be an intermediate module 7 which contacts on one side the microsieve 6 and on the other side the outlet module 8. All modules and components, e.g. spacer and microsieve, are attached to each other in a removably manner, i.e. they can be easily separated from each other for assembly and disassembly of the device. However, if desired, some or all of the modules and components of the cell separation devices described herein can be permanently fixed to each other, either by manufacturing a device made of one single piece or two or three different pieces which are then fixed, such as glued, together.

If attached to each other in a removable manner as illustrated in FIG. 5, the modules can comprise openings 301, 302, 303 for inserting fixation means, such as screws or pins. As for the device illustrated in FIGS. 1 to 4, those openings can also form part of connectors attached to the outer rim of the modules 4, 7, 8. In alternative, the way of connecting the components together via openings 301 which are integrated in the module itself can also be used in the embodiment illustrated in FIGS. 1 to 4.

The cell separation devices described herein can further comprise positioners which ensure a defined orientation of the modules 4, 7, 8 relative to each other in the assemble state. Therefore, the modules can comprise receiving openings 400, 402 for receiving the positioners 401, 403 which fit into the receiving openings 400, 402. The positioners can have any shape as long as they allow attaching the inlet module 4, outlet module 8 and intermediate module 7 together in a specific orientation. In one embodiment, the positioners 401, 403 have a dome-shape while the receiving openings 400, 402 have the corresponding shape to receive the dome-shaped positioners 401, 403. The postioners are also designed for self-guiding the intermediate module 7 on a microscope stage for consistent imaging and detection. The positioners of the intermediate module 7 can thus ensure that the intermediate module 7 is positioned always in the same manner under a detection device, such as a microscope, to ensure imaging always the same pre-defined area. The positioners can be arranged at the outer perimeter of the inlet module 4, outlet module 8 and intermediate module 7.

Furthermore, like the outlet module 3 in the embodiment shown in FIGS. 1 to 4, the intermediate module 7 in the embodiment illustrated in FIG. 5 can comprise a recess 72 which is adapted to house either the microsieve 6 alone or the microsieve 6 and the optional spacer 5 together. The characteristics of the recess 72 are the same as for the recess 31 in the outlet module 3 of the device illustrated in FIGS. 1 to 4.

The recess 72 comprises a through-hole 71 through which the filtered sample which already passed through the microsieve flows towards the outlet 36 of the outlet module 8. The negative pressure or suction force, which can be applied to the outlet of the outlet module via the orifice 81 of the outlet module, drives the liquid sample through the cell separating device. The through-hole 61 can have the same size as the filtration area 61 or can be larger in size and can exceed the size of the filtration area 61. The through-hole can be positioned in direct line with the openings of the inlet module, spacer (if present) and filtration are 61 of the microsieve or can be arranged anywhere else at the surface of the intermediate module 7. If present, the through-hole can be arranged somewhere within the area confined by the recess 71 or can be arranged in the center of the recess 72 as illustrated in FIG. 5. A central position ensures lesser fluid resistance which would be caused if the liquid has to flow around corners or pass a certain distance along a horizontal plane instead of a straight vertical path.

The intermediate module 7 can be removed from the cell separation device illustrated in FIG. 5 to be placed under a microscope for imaging cells isolated by the microsieve 6. For neither this purpose the microsieve 6 nor the spacer 5 (if present) need to be removed. The cells located at the surface of the microsieve 6 which did not pass through the pores of the microsieve 6 can be analyzed via a microscope through the opening 51 of the spacer 5. Other than in the embodiment illustrated in FIGS. 1 to 4, only the intermediate module needs to be removed and placed under a microscope. For microscopic analysis the thickness of the intermediate module 7 is selected to be thin enough to be easily arrangeable on the stage of the microscope directly under an objective.

The outlet module 8 may comprise a protrusion (not shown in FIG. 5) which is a continuation of the outlet 36 towards the intermediate module 7. Thus, this protrusion would be dimensioned to fit into the through-hole of the intermediate module. The protrusion would abut the wall of the through-hole 71 and can ensure that fluid flowing from the intermediate module 7 to the outlet module 8 does not spread in the contact area between intermediate module 7 and outlet module 8.

The outlet 36 of the outlet module 8 can extend through the entire body of the outlet module but can also lead to a chamber 37 which forms part of the outlet module. The chamber 37 can comprise the orifice 81 of the outlet module. This chamber can have any shape. In one embodiment, the chamber 37 is adapted to provide a space to fit the outlet module 8 to a container connectable to the outlet module 8, such as a flask as illustrated for example in FIG. 13. Such a chamber 38 can also be comprised in the embodiment of the device illustrated in FIG. 1. Such a chamber can comprise a thread or other means known in the art for fitting the cell separation devices referred to herein to a container.

Like for the cell separation device illustrated in FIGS. 1 to 4, the cell separation device illustrated in FIG. 5 can also comprise trenches and isolation material running around the outer perimeter of the modules 4, 7, 8. Water repellent isolation materials can be fit into those trenches to provide a water tight closure when the modules 4, 7, 8 are assembled. O-ring gaskets made of a polymeric material can be used as an isolation material.

In the devices described herein it is possible to integrate multiple intermediate modules 2 and 7 and multiple microsieves 6 which allow cells of different sizes and also other components which might be comprised in the liquid sample other than cells to be separated from each other. For example, with regard to the separation device illustrated in

FIG. 5, it is possible to stack two, three or even more intermediate modules 7 onto each other each carrying a microsieve 6 with a pore size adapted to filter a cell or component of specific size out of the liquid sample. In case multiple microsieves are used, the first microsieve would comprise the largest pore size while the pore size narrows down with every following intermediate module carrying a microsieve. With respect to the embodiment illustrated in FIGS. 1 to 4, additional intermediate modules 2 and outlet modules 3 might need to be included. To minimize the overall size of the device the addition outlet modules could be provided with an orifice 32 and side wall 33. Also, the outlet 35 of the outlet module could extend into a protrusion like the protrusion 13 of the inlet module to abut or almost abut the surface of the following intermediate module around the opening 21.

In general, an intermediate module 2, 7 can have a thickness of between about 1 to 5 mm and is thus considerable thinner than the thickness of the inlet module 1, 4 and the outlet module 3, 8. The dimensions of the different components of the devices for separating cells can be freely adapted to the necessary application, i.e. there are no specific limits. In one embodiment, the maximal dimension of an inlet module, outlet module and intermediate module in the horizontal direction is between about 2 to 8 cm or between about 2 to 5 cm. The maximal thickness of the devices for separating cells can be between about 5 to 15 cm.

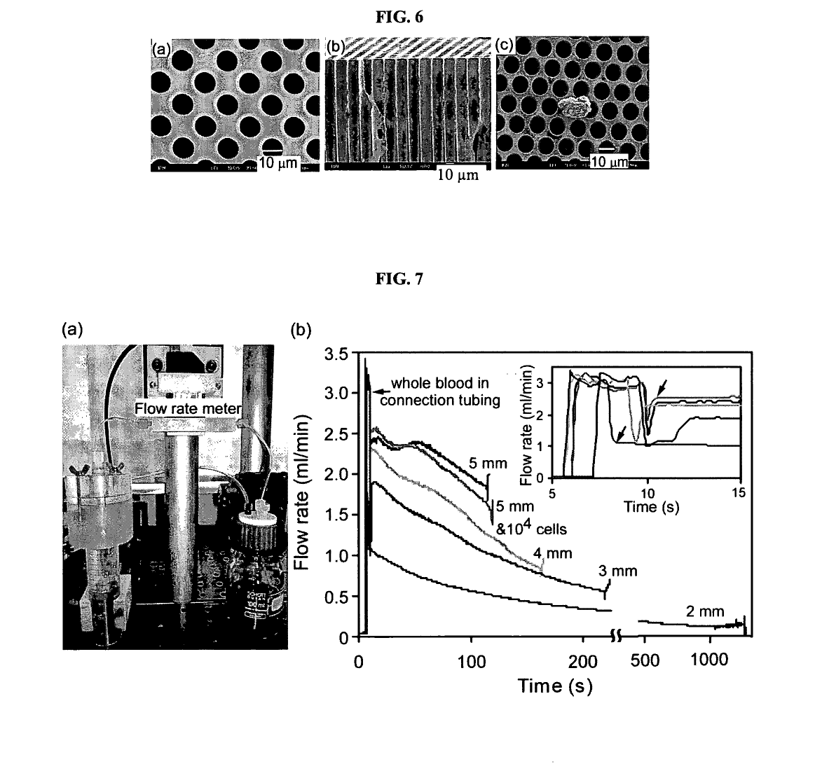

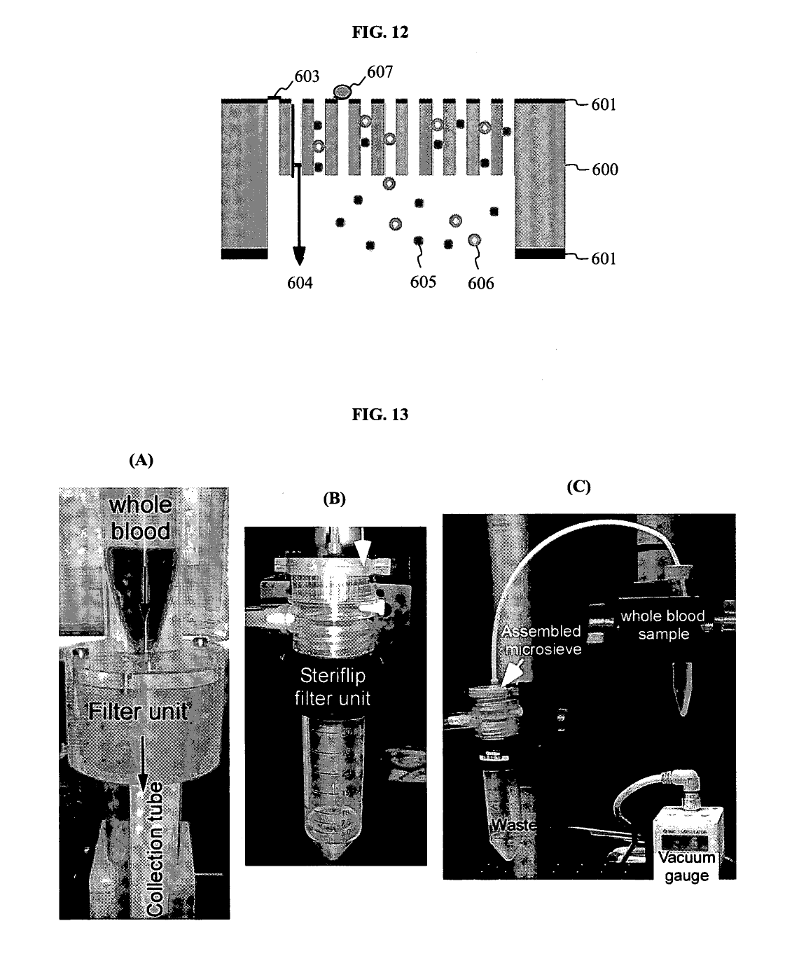

In general, the microsieves used herein work according to the principal illustrated in FIG. 12. A microsieve comprises micropores 603 having a size adapted to separate cells 607 of a defined size, such as circulating tumor cells (CTCs) from a liquid sample, such as whole blood. While the cells are retained at the surface of the microsieve, other components pass through the filter. The microsieve can have a filtration region which is defined by the size of the filtration area and the length of the channels formed by each pore. The channels formed by the pores in the microsieve are illustrated exemplarily in FIG. 6(B). The length of the pores can be the same or shorter than the overall thickness of the microsieve as illustrated for example in FIG. 12. The thickness of the microsieve can be between about 50 μm to about 1000 μm or about 150 μm. In contrast, the length of the pores, i.e. the pore channel length can be between about 50 μm to about 250 μm or between about 50 μm to about 150 μm. In one embodiment the thickness is at least 50 μm or at least 100 μm or at least 150 μm. The length of the channel 604 formed by the micropores through the microsieve 6 has a length of at least 50 μm. The specific length of these channels provides a fluidic flow condition that is close to the conditions of in vivo blood flow in arterioles. In addition, the viscosity of blood inside a channel further depends on the pore diameter 604, which has a minimum around channel diameter of 10 μm. The combination of these effects provides a high extraction efficiency. Known microsieves combine vacuum force or pressure and microsieve dimensions (pore diameter, channel length) randomly and thus do not achieve this effect.

For example, the following Table 1 illustrates the fluidic parameters which were obtained using any of the devices desribed herein for filtering a fluidic sample, in this example whole blood for separating cells dispersed in the whole blood sample.

TABLE 1
Calculated fluidic parameters of microsieve filtration.

| Microsieve | Initial flow rate | Velocity | Shear rate | |
|---|---|---|---|---|
| diameter (mm)*² | (Q; ml/min) | (Vₘ; mm/s) | (τ, s⁻¹) | Re |
| 5 | 2.49 | 3.35 | 335 | 0.009 |
| 5*¹ | 2.64 | 3.55 | 355 | 0.010 |
| 4 | 2.38 | 5.01 | 501 | 0.014 |
| 3 | 1.98 | 7.41 | 741 | 0.021 |
| 2 | 1.16 | 9.26 | 976 | 0.028 |

Notation:
Re is the Renolds number, Re = ρVₘa/μ_{b,} where Vₘ is the velocity, μ_{b} = 3.8 cps is the bluk viscosity of blood at 45% hematocrit, ρ = 1060 Kg/m³ is the blood density and a is the pore diameter (10 μm).
Vₘ = Q/AΦ, where Q is the initial volume flow rate at the point of blood contacting with microsieve, A is the membrane area, and Φ = 0.63 is the microsieve porosity with 10-μm pore diameter and 12-μm pitch.
τ = Vₘ/a is the shear rate.
*¹With 10⁴ of HepG2 cells
*²Microsieve diameter (mm) refers to the filtration area available for filtration of the liquid sample through any of the devices described herein.

The maximal horizontal extension of the microsieve or in case of a round microsieve the diameter thereof, can be between about 1 mm to about 3 cm or about 1.5 cm. The micropores can be spaced apart from each other in a uniform pattern, i.e. they form a regular matrix as for example illustrated in FIGS. 6(A) and 6(C). In such a uniform pore matrix the maximal distance from one micropore to another micropore can be between about 2 μm to about 100 μm. In one embodiment, the distance is about 12 μm. As already mentioned, the pore size depends on the cell to be filtered. In general, the size of the micropores is between about 2 μm to about 20 μm or about 10 μm.

For example, circulating tumor cells (CTCs) which can be found in blood of patients can have a size of between about 15 to 40 μm whereas leukocytes have a size of 10 μm. Thus, with reference to FIG. 12, to extract such CTCs the pores should be big enough 603 to let leucocytes 606 and other smaller blood components, such as erythrocytes 605 pass through the pores of the microsieve 6 but small enough to filter out CTCs 607.

A microsieve can be made of any material, such as glass, silica, metal mesh, and SU-8 epoxy-based negative photoresist.

The present invention also refers to a system for cell separation. In addition to a device described herein, such a system can comprise a container connected to the outlet module of the device. As described above, the outlet module of the devices described herein can be adapted to be connectable to such containers, such as flasks. A device fitted onto the opening of a flask is illustrated for example in FIGS. 13(A) to (C). Furthermore, such a system can also include an apparatus for exerting a negative pressure at the outlet of the outlet module 3, 8. A vacuum force applied can be between about 2 to 40 kPa. In one embodiment, the vacuum force is about 10 kPa or 20 kPA or 30 kPA. An apparatus for exerting a negative pressure can be for example a vacuum pump.

The system can further include a source for holding a liquid, such as another container or even a container which is connected to a pump which is actively pumping the liquid towards the cell separation device for further increasing the filtration speed. It might also be possible to actively pump the fluid sample through the device rather than driving it through the device by application of a negative pressure at the outlet module thereof.

For the devices referred to herein filtration efficiency for example for CTCs from blood of about 94% has been shown. Furthermore, the average filtration speed for whole blood can be between about 1 to 3 ml/min or between about 1.4 to 2.5 ml/min, depending on the size of the effective filtration area on the microsieve (see for example FIG. 7). For example, in FIG. 7 the flow rate of whole blood which has a higher viscosity than e.g. an aqueous solution, such as PBS buffer, was determined on the basis of different round filtration areas having diameters of between about 2 to 5 mm. The flow rate has been calibrated with rabbit whole blood. In comparison, other microfluidic devices have much lower flow rates which are about <0.1 ml/min.

The present invention also refers to a method of separating cells of a defined size, such as CTCs, from a liquid sample. Examples of CTCs include, but are not limited to HepG2, responsible for liver carcinoma; MCF-7, responsible for breast carcinoma; CD4⁺ T-cells for HIV; fetal cells for prenatal testing. Other cells that can be filtered include, but are not limited to cancer cells or cancer stem cells from lysed cancer tissue, cells comprised in a urine sample, or enrichment of cells from cell culture medium. For this method a liquid sample, such as whole blood, or urine, or culture medium, or lysed tissue solution is introduced into and filtered through any of the devices described herein. After filtration, the isolated cells can be either directly examined with optical detection devices, such as a microscope and/or can be removed from the microsieve for further examination and treatment.

This simple, low cost, and highly efficient devices and the respective methods of using them can have implications in advancing both cancer biology research and clinical cancer management, including the detection, diagnosis and monitoring of cancer.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Manufacture of Microsieve

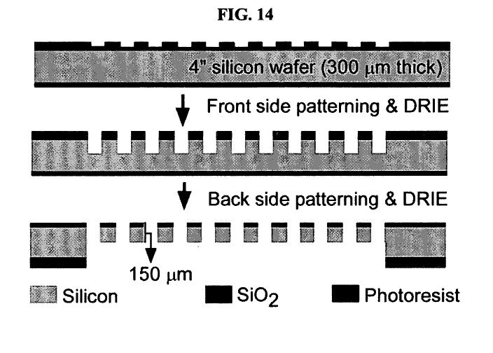

An exemplary microsieve filter was fabricated by using deep reactive ion etching (DRIE) on 4″ diameter silicon wafer (FIG. 14) or by using a commercial glass capillary array. In brief, the microsieve filter was fabricated by using single mask lithography, and a dual side reactive ion etching process on 4″-diameter silicon wafer (300-μm thick) with the pattern of the microsieve (pore size e.g. of 10 μm in diameter with 15 μm pitch) created by an 100 or 150 μm deep silicon etch using a photoresist/oxide hard mask, and with the membrane area of microsieve filter defined by a subsequent SF₆-based isotropic back etch.

Filtration Area

The optimal area of a microsieve filter was tested prior to use (FIG. 7) with a liquid flow meter (SLG1600-20, Sensirion) connected between the device and an air-vented whole blood reservoir containing fresh rabbit whole blood (5 ml) with anticoagulant EDTA. The flow rate variation was monitored as the microsieve filter was subjected to a vacuum force of 5 kpa (in general 5 to 40 kPa is usable). FIG. 7 shows the microsieve's diameter (2-5 mm) and the flow rate as functions of time. The results indicate that the devices described herein have a very low fluidic resistance, and can be applied directly with undiluted whole blood.

FIG. 6 shows the highly uniform pore structure and smooth through-hole surface of the densely packed pore array (such as >4000 pores/mm²). Unlike in-plane microsieves which have limited cell extraction speed due to the high fluidic resistance of the lateral fluidic structure (few-cm channel length, and ˜100 μm channel height), the vertical microsieve has much lower fluidic resistance (pore or channel length 604 of 150 μm with pore opening ≧35% of device area), allowing fast CTCs isolation. FIGS. 1 and 5 show the device assembly.

In FIG. 5 the microsieve 6 is sandwiched between the inlet module 4 and the intermediate module 7 with integrated fluid connectors 41, 71. The effective pore area or filtration area 61 is self-defined by the opening of inlet connection 41. In a further embodiment, the devices can be further integrated with a Millpore Steriflip® filter unit to provide a ready-to-use component (FIG. 13(B)).

Separation of Cells

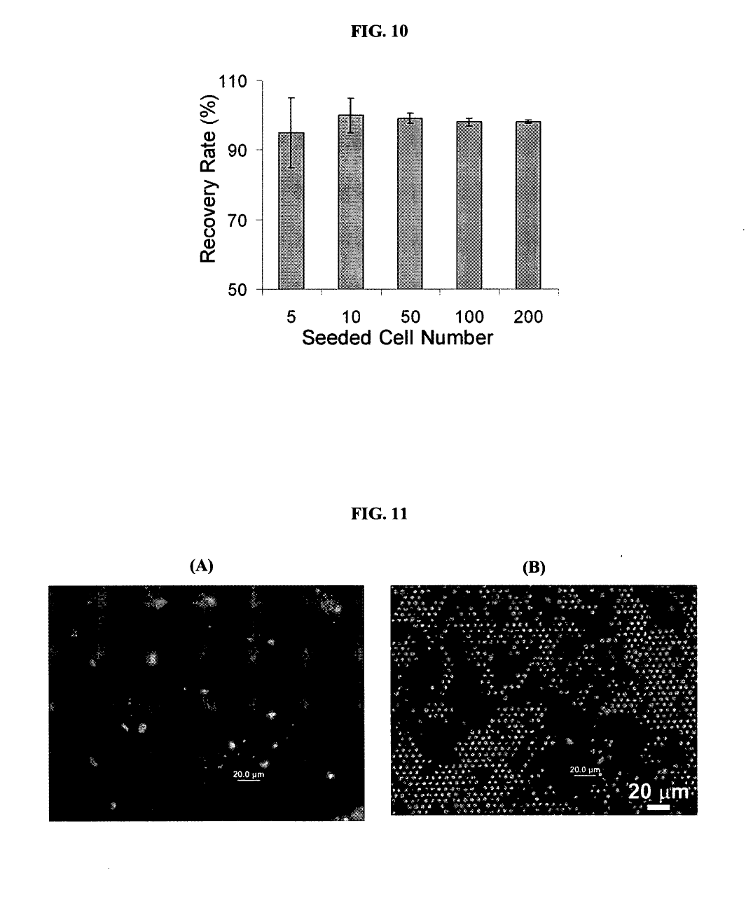

Devices as shown in FIG. 1 or 5 were used to separate CTCs, here HepG2 cells, from whole blood samples. The microsieve used in the device utilizes the distinct morphology and size difference of cancer cells (diameter 12 to 40 μm) and leukocytes (diameter ≦10 μm but not 0 μm) to extract the CTCs from whole blood sample. The microsieve used in the devices contains densely packed pore arrays (>4000 pores/mm²) with a pore diameter of 10 μm (603 in FIG. 12). Such a microsieve is capable to effectively retain >94% of various cancer cells (FIG. 10). The SEM image of HepG2 cell (live carcinoma) isolated on the microsieve clearly shows the relatively dimension of the cancer cell and the microsieve (FIG. 6(C)).

FIG. 13(C) shows the experimental setup for isolation of CTCs in another example. The fluidic flow of whole blood was controlled by a respiration force connected to the integrated microsieve filter unit through a vacuum gauge (ITV2091-212BL5, SMC Phenumatics). The filtrated waste of whole blood sample was collected by a centrifuge tube. This system is very simple compared with existing CTCs isolation methods using spare fused pores membrane filter, antibody conjugated lab-chip devices, antibody conjugated magnetic beads, and microfabricated parylene membrane filter. Successful CTCs isolation has been demonstrated on both of cancer cell lines (HepG2: liver carcinoma; MCF-7: breast carcinoma; 1-50 cells/ml diluted in phosphate buffered saline) and whole blood sample of rat with lung cancer (FIG. 11). Captured CTCs were stained with DAPI (nucleus) and EpCAM (epithelial cell marker), and manually counted with a fluorescence microscope. The extraction time in a whole blood sample with a volume of 5 ml was less than 15 minutes when the round filtration area had a diameter of 2 mm. In one example, 5 ml of blood have been filtered through the cell separation device within 2 mins.

In another example, to determine the efficiency of capture of CTCs, such as HepG2, cultured liver cancer cells (HepG2/GFP) were spiked into rabbit whole blood at 10 to 100 cell/ml and the spiked cancers cells were captured using a microsieve as described above.

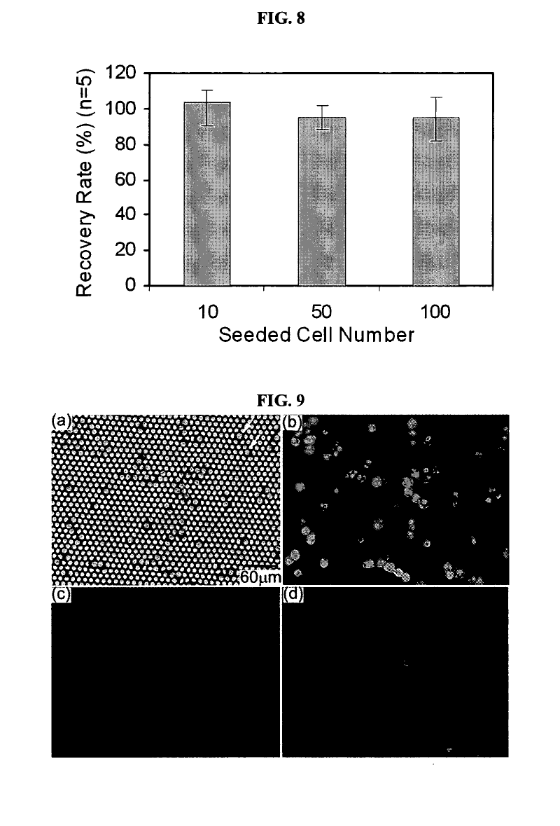

HepG2/GFP cell line (shown in green with emission peak at ˜509 nm) was established by transformation HepG2 cell with GFP reporter plasmid, which allows visually counting the cancer cells under a fluorescent microscope. Prior to filtration, the HepG2/GFP cells were fixed following standard fixation procedures, and counted manually on hemocytometers (Hausser Scientific, Horsham, Pa., USA). The nucleus of HepG2 were dyed with DAPI and shown in blue (FIGS. 9 and 11) (emission peak at ˜460 nm) in the fluorescence images, while on the cell surface a green fluorescent protein (GFP) was expressed which is shown in green (˜509 nm). The captured CTCs were manually counted with an up-right Lecia DM 5000B fluorescence microscope with the software IMAGE-PRO 6.0 (Media Cybernetics, USA). The gray level images were artificially colored with the software to match the respective colors of the filters used. Furthermore, FIG. 8 shows the recovery rate of HepG2 (10-100 cells) spiked in 1-ml rabbit whole blood sample. The average recovery rate is >90%.

### LIST OF REFERENCE NUMBERS

1, 4 Inlet module
2, 7 Intermediate module
3, 8 Outlet module
5 Spacer
6 Microsieve
11, 42 Opening of inlet
12, 23 Trench
13 Protrusion
14 Inlet of inlet module
21 Through-hole of intermediate module
22 Cone or funnel of intermediate module
24 Extension of intermediate module
31 Recess
32 Orifice
33 Side wall of outlet module
35, 36 Outlet of outlet module
37, 38 Chamber
41 Inlet of inlet module
51 Through-hole of spacer
61 Filtration area
71 Through-hole of intermediate module
72 Recess of intermediate module
81 Orifice of outlet module
201, 202, 203 Connector
205, 206, 207, 301, 302, 303 Opening for fastening means
400, 402 Receiving opening for positioner
401, 403 Positioner
600 Silicon wafer
601 SiO₂ layer
603 Indicator for pore diameter
604 Indicator for length or depth of pore
605 Erythrocytes
606 Leucocytes
607 Circulating tumor cell (CTC)

1. A device for separating cells of a defined size from a sample, wherein the device comprises:
an inlet module having an inlet;
an outlet module having an outlet;
an intermediate module having a through-hole; wherein the intermediate module is arranged between the inlet module and the outlet module;
a microsieve having micropores for retaining cells of a defined size and being arranged between the inlet module and the intermediate module; and
wherein the inlet module, the outlet module, and the intermediate module are removably and fluidly connected to each other; and
wherein a channel formed by each of the micropores of the microsieve through the microsieve has a length of about 50 μm to about 250 μm; and
wherein a diameter of each of the micropores is between about 2 μm to about 20 μm.

2. A device for separating cells of a defined size from a sample, wherein the device comprises:
an inlet module having an inlet;
an outlet module having an outlet;
an intermediate module having a through-hole and being arranged between the inlet module and the outlet module;
a microsieve having micropores for retaining cells of a defined size;
wherein the inlet module, the intermediate module and the outlet module are removably and fluidly connected to each other;
wherein the microsieve is arranged between the intermediate module and the outlet module; and
wherein a channel formed by each of the pores of the microsieve through the microsieve has a length of about 50 μm to about 250 μm; and
wherein a diameter of each of the micropores is between about 2 μm to about 20 μm.

3. The device of claim 2, wherein the inlet module comprises a protrusion which is arranged at the side of the inlet module opposite the inlet and wherein the protrusion is a continuation of the inlet and is adapted to contact the surface of the intermediate module around the through-hole of the intermediate module when assembled.

4. The device of claim 2, wherein the intermediate module has the shape of a cone and wherein the through-hole of the intermediate module is arranged at the bottom of the cone.

5. The device of claim 4, wherein the tip of the cone is facing the microsieve.

6. The device of claim 2, wherein the intermediate module defines a filtering area on the microsieve and wherein the size of the filtering area is defined by the size of the through-hole of the intermediate module.

7. The device of claim 2, wherein the outlet module comprises a top surface having a recess arranged in the top surface;
wherein the recess is adapted for holding the microsieve, and wherein the recess comprises the outlet of the outlet module.

8. The device of claim 7, wherein the outlet in the recess of the outlet module is positioned opposite the opening of the intermediate module.

9. The device of claim 8, wherein the outlet module comprises a side wall with an orifice, wherein the orifice is positioned outside the recess and wherein the orifice is connectable to a device exerting a negative pressure.

10. (canceled)

11. The device of claim 1, wherein the thickness of the microsieve is between about 50 μm to about 1000 μm or about 150 μm.

12.-15. (canceled)

16. The device of claim 1, wherein the maximal distance from the center of one micropore to the center of another micropore is between about 2 μm to about 100 μm or about 12 μm.

17.-18. (canceled)

19. The device of claim 1, further comprising a spacer having a through-hole and being arranged between the microsieve and the inlet module; and
wherein the spacer defines a filtering area on the microsieve and wherein the size of the filtering area is defined by the size of the through-hole of the spacer.

20. The device of claim 1, wherein the intermediate module comprises a recess for holding the microsieve.

21. (canceled)

22. The device of claim 19, wherein the filtering area has a maximal dimension of between about 0.5 to 20 mm or between about 2 to 5 mm.

23. The device of claim 1, wherein the outlet module is adapted to exert a negative pressure at the outlet thereof.

24. (canceled)

25. A system comprising:
a device, for separating cells of a defined size from a sample, wherein the device comprises:
an inlet module having an inlet;
an outlet module having an outlet;
an intermediate module having a through-hole and being arranged between the inlet module and the outlet module;
a microsieve having micropores for retaining cells of a defined size;
wherein the inlet module, the intermediate module and the outlet module are removably and fluidly connected to each other; and
wherein a channel formed by each of the pores of the microsieve through the microsieve has a length of about 50 μm to about 250 μm; and
wherein a diameter of each of the micropores is between about 2 μm to about 20 μm,
a container connected to the outlet module of the device, and
an apparatus for exerting a negative pressure which is connected to an orifice of the device adapted to be connectable to the apparatus for exerting a negative pressure.

26. (canceled)

27. A method of separating cells of a defined size from a liquid sample comprising:
filtering a liquid sample suspected to comprise a cell to be separated through an inlet of a device of for separating cells of a defined size from a sample, wherein the device comprises:
an inlet module having an inlet;
an outlet module having an outlet;
an intermediate module having a through-hole; wherein the intermediate module is arranged between the inlet module and the outlet module;
a microsieve having micropores for retaining cells of a defined size and being arranged between the inlet module and the intermediate module; and
wherein the inlet module, the outlet module, and the intermediate module are removably and fluidly connected to each other; and
wherein a channel formed by each of the micropores of the microsieve through the microsieve has a length of about 50 μm to about 250 μm; and
wherein a diameter of each of the micropores is between about 2 μm to about 20 μm.

28.-32. (canceled)

33. The device of claim 2, wherein the outlet module is adapted to exert a negative pressure at the outlet thereof.

34. A system comprising:
a device for separating cells of a defined size from a sample, wherein the device comprises:
an inlet module having an inlet;
an outlet module having an outlet;
an intermediate module having a through-hole and being arranged between the inlet module and the outlet module;
a microsieve having micropores for retaining cells of a defined size;
wherein the inlet module, the intermediate module and the outlet module are removably and fluidly connected to each other;
wherein the microsieve is arranged between the intermediate module and the outlet module; and
wherein a channel formed by each of the pores of the microsieve through the microsieve has a length of about 50 μm to about 250 μm; and
wherein a diameter of each of the micropores is between about 2 μm to about 20 μm,
a container connected to the outlet module of the device, and
an apparatus for exerting a negative pressure which is connected to an orifice of the device adapted to be connectable to the apparatus for exerting a negative pressure.

35. A method of separating cells of a defined size from a liquid sample comprising:
filtering a liquid sample suspected to comprise a cell to be separated through an inlet of a device for separating cells of a defined size from a sample, wherein the device comprises:
an inlet module having an inlet;
an outlet module having an outlet;
an intermediate module having a through-hole and being arranged between the inlet module and the outlet module;
a microsieve having micropores for retaining cells of a defined size;
wherein the inlet module, the intermediate module and the outlet module are removably and fluidly connected to each other;
wherein the microsieve is arranged between the intermediate module and the outlet module; and
wherein a channel formed by each of the pores of the microsieve through the microsieve has a length of about 50 μm to about 250 μm; and
wherein a diameter of each of the micropores is between about 2 μm to about 20 μm.
